(19) United States
(12) Patent Application Publication
(10) Pub. No.: US 2008/0227999 A1
(43) Pub. Date: Sep. 18, 2008

(54) Heterogeneous Supported Catalytic Carbamate Process

(76) Inventor: David C. Molzahn, Midland, MI (US)

Correspondence Address: The Dow Chemical Company, Intellectual Property Section, P.O. Box 1967, Midland, MI, 48641-1967, US

(21) Appl. No.: 11/994,510

(22) PCT Filed: Jul. 17, 2006
(86) PCT No.: PCT/US2006/027825

§ 371 Date: Jan. 3, 2008

Related U.S. Application Data
(60) Provisional application No. 60/701,049, filed on Jul. 20, 2005.

(51) Int. Cl. C07C 269/00; C07C 269/04; C07C 263/00

(52) U.S. Cl. 560/24; 560/341

(57) ABSTRACT

A process for the preparation of aromatic carbamates comprising contacting one or more organic carbonates with an aromatic amine or urea in the presence of a catalyst and recovering the resulting aromatic carbamate product, characterized in that the catalyst is a heterogeneous catalyst comprising a Group 12-15 metal compound supported on a substrate.

### BACKGROUND OF THE INVENTION

The present invention relates to a process for preparing carbamates from aromatic amines or ureas in high yields and efficiencies. The products include aromatic carbamates which are usefully employed in the manufacture of isocyanates, such as toluene diisocyanate and other commercially valuable compounds.

The reactions of organic carbonates with aromatic amines to form carbamates are well known. Numerous patents and articles describing this chemistry are in existence. Examples include: U.S. Pat. Nos. 4,268,683, 4,268,684, 4,381,404, 4,550,188, 4,567,287, 5,091,556, 5,688,988, 5,698,731, and 6,034,265 as well as EP-A-048,371. Non-patent literature sources include: “Synthesis of Toluene Diisocyanate with Dimethyl Carbonate Instead of Phosgene”, Zhao Xin-Qiang, et al., Petrochemical Technology, 28, 614 (1999).

While providing satisfactory routes to the desired reaction products the foregoing techniques generally employ homogeneous catalysts which require subsequent purification of the resulting products to remove soluble metal values before continued processing or use. In addition current processes involve mixing of the homogeneous catalyst and reaction followed by separation and recovery of the catalysts, usually from a solution with the reaction products. The need for such a separation step increases the cost of current production methods. Accordingly, there remains a desire in the art to provide a heterogeneous catalyst and associated process for the production of the foregoing valuable industrial materials in an improved process.

### SUMMARY OF THE INVENTION

According to one embodiment of the present invention there is provided an improved process for the preparation of carbamates comprising contacting one or more organic carbonates with an aromatic amine or urea in the presence of a catalyst and recovering the resulting product, characterized in that the catalyst is a heterogeneous catalyst comprising a Group 12-15 metal compound supported on a substrate.

In another embodiment of the invention the foregoing process is included as one step in a multiple step process for the formation of an isocyanate from an aromatic amine and carbon monoxide, said process comprising the steps of:

1) contacting a dialkyl carbonate with an aromatic amine in the presence of a heterogeneous catalyst comprising a Group 12-15 metal compound supported on a substrate to form an alkylcarbamate and an alcohol;

2) thermally decomposing the alkylcarbamate to form an aromatic isocyanate compound and an alcohol;

3) contacting the alcohol from step 1) and/or 2) with carbon monoxide under conditions to reform the dialkyl carbonate; and

4) recycling the dialkyl carbonate formed in step 3) for use in step 1).

Because the process uses a heterogeneous catalyst for the formation of the carbamate product or intermediate, purification of the product to remove metal values may be avoided and unit operations involving a fixed catalyst bed may be employed, thereby achieving improved process efficiencies and reduced cost.

### DETAILED DESCRIPTION OF THE INVENTION

All references to the Periodic Table of the Elements herein shall refer to the Periodic Table of the Elements, published and copyrighted by CRC Press, Inc., 2003. Also, any references to a Group or Groups shall be to the Group or Groups reflected in this Periodic Table of the Elements using the IUPAC system for numbering groups. Unless stated to the contrary, implicit from the context, or customary in the art, all parts and percents are based on weight. For purposes of United States patent practice, the contents of any patent, patent application, or publication referenced herein are hereby incorporated by reference in their entirety (or the equivalent US version thereof is so incorporated by reference) especially with respect to the disclosure of synthetic techniques, definitions (to the extent not inconsistent with any definitions provided herein) and general knowledge in the art.

The term “comprising” and derivatives thereof is not intended to exclude the presence of any additional portion, component, step or procedure, whether or not the same is disclosed herein. In order to avoid any doubt, all compositions claimed herein through use of the term “comprising” may include any additional additive, adjuvant, or compound unless stated to the contrary. In contrast, the term, “consisting essentially of” excludes from the scope of any succeeding recitation any other portion, component, step or procedure, excepting those that are not essential to operability. The term “consisting of” excludes any portion, component, step or procedure not specifically delineated or listed. The term “or”, unless stated otherwise, refers to the listed members individually as well as in any combination.

As used herein with respect to a chemical compound, unless specifically indicated otherwise, the singular includes all isomeric forms and vice versa (for example, “hexane”, includes all isomers of hexane individually or collectively). The terms “compound” and “complex” are used interchangeably herein to refer to organic-, inorganic- and organometal compounds. The term, “atom” refers to the smallest constituent of an element regardless of ionic state, that is, whether or not the same bears a charge or partial charge or is bonded to another atom. The term “heteroatom” refers to an atom other than carbon or hydrogen. Preferred heteroatoms include: F, Cl, Br, N, O, P, B, S, Si, Sb, Al, Sn, As, Se and Ge.

As used herein the term “aromatic” refers to a polyatomic, cyclic, conjugated ring system containing (4δ+2) π-electrons, wherein δ is an integer greater than or equal to 1. The term “fused” as used herein with respect to a ring system containing two or more polyatomic, cyclic rings means that with respect to at least two rings thereof, at least one pair of adjacent atoms is included in both rings. The term “aryl” refers to a monovalent aromatic substituent which may be a single aromatic ring or multiple aromatic rings which are fused together, linked covalently, or linked to a common group such as a methylene or ethylene moiety. The aromatic ring(s) may include phenyl, naphthyl, anthracenyl, and biphenyl, among others.

“Substituted aryl” refers to an aryl group in which one or more hydrogen atoms bound to any carbon is replaced by one or more functional groups such as alkyl, substituted alkyl, cycloalkyl, substituted cycloalkyl, heterocycloalkyl, substituted heterocycloalkyl, halogen, alkylhalos (e.g., CF₃), hydroxy, amino, phosphido, alkoxy, amino, thio, nitro, and both saturated and unsaturated cyclic hydrocarbons which are fused to the aromatic ring(s), linked covalently or linked to a common group such as a methylene or ethylene moiety. The common linking group may also be a carbonyl as in benzophenone or oxygen as in diphenylether or nitrogen as in diphenylamine.

The improved process of the present invention for the formation of carbamates from aromatic amines or ureas can be illustrated by the following schematic representations:

Ar(NRH)ᵣ+R′OC(O)O [symbol] Ar(NRC(O)OR′)ᵣ+R′OH

Ar(NRC(O)NRR″)ᵣ+R′OC(O)O [symbol] Ar(NRC(O)OR)ᵣ+R″NRC(O)OR′,

wherein,

Ar is an aromatic or substituted aromatic group having a valency of r,

R independently each occurrence is hydrogen, alkyl, or aralkyl,

R′ independently each occurrence is alkyl or two R′ groups together are alkylene, and

R″ independently each occurrence is R or Ar.

Examples of suitable aromatic groups include those having the formulae:

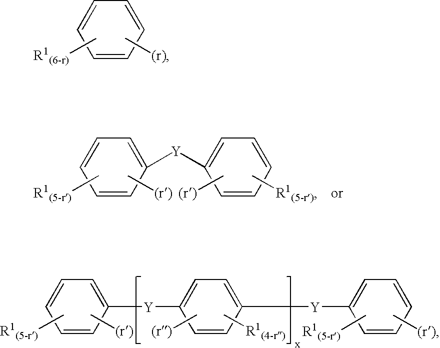

wherein R¹ independently each occurrence is hydrogen, halo, hydrocarbyl, inertly substituted hydrocarbyl, trihydrocarbylsilyl, trihydrocarbylsilylhydrocarbyl, or hydrocarbyloxy,

r is an integer greater than or equal to 1 which is equal to the valency of the aromatic group,

r′ individually each occurrence is an integer greater than or equal to 0 with the proviso that the sum of all r′ present (if no r″ is present) equals r,

r″ individually each occurrence is an integer greater than or equal to 0 with the proviso that where r″ is present, the sum x(r″)+all r′ equals r,

Y is selected from the group consisting of —O—, —CO—, —CH₂—, —SO₂—, —NR¹C(O)—, and a single bond, and

x is an integer greater than or equal to 0 indicating the number of repeating groups in the aromatic radical.

The skilled artisan will appreciate that a mixture of the foregoing aromatic groups may be present in the aromatic amine or urea compounds used in the present invention.

The term “hydrocarbyl” means the monovalent radical obtained by removing one hydrogen atom from a parent hydrocarbon, preferably having from 1 to 8 carbon atoms. Illustrative hydrocarbyl groups, include alkyl, such as methyl, ethyl, propyl, butyl, pentyl, hexyl, or octyl, including all isomeric forms thereof; alkenyl, such as vinyl, allyl, butenyl, pentenyl, hexenyl, or octenyl, including all isomeric forms thereof; aralkyl, such as benzyl, phenethyl, or methylbenzyl, including all isomeric forms thereof; aryl such as phenyl, tolyl, xylyl, anthracenyl, or diphenyl, including all isomeric forms thereof; cycloalkyl such as cyclobutyl, cyclopentyl, cyclohexyl, cycloheptyl, or cyclooctyl, including all isomeric forms thereof; and cycloalkenyl such as cyclopentenyl, cyclohexenyl, cycloheptenyl, or cyclooctenyl, including all isomeric forms thereof.

The term “inert substituent” means any radical other than hydrocarbyl that does not interfere with the process in accordance with the present invention. Illustrative of such substituents are halo, such as chloro, bromo, fluoro or iodo; nitro; alkoxy, such as methoxy, ethoxy, propoxy, butoxy, pentyloxy, hexyloxy, heptyloxy, or octyloxy, including isomeric forms thereof; alkylmercapto, such as methylmercapto, ethylmercapto, propylmercapto, butylmercapto, pentylmercapto, hexylmercapto, heptylmercapto, or octylmercapto, including all isomeric forms thereof; cyano; and combinations of the foregoing. Preferred inert substituents are those containing from 1 to 8 carbon or carbon+heteroatoms.

It is to be understood that when a polyamine reactant is employed, the product would be the corresponding polycarbamate. Similarly, when a polyurea reactant is employed the product would be the corresponding polycarbamate or mixed polycarbamate. Detailed descriptions of the respective reagents and products are previously disclosed in U.S. Pat. Nos. 4,268,683; 4,268,684, 4,395,565, 4,550,188 and 4,567,287.

Examples of suitable aromatic amine reagents for use herein include: aniline, p-methoxyaniline, p-chloroaniline, o-, m- or p-toluidine, 2,4-xylidine, 2,4-, and 2,6-toluenediamine, m- or p-phenylenediamine, 4,4′-diphenylenediamine, methylenebis(aniline) including 4,4′-methylenebis(aniline), 2,4′-methylenebis(aniline), 4,4′-oxybis(aniline), 4,4′-carbonylbis(aniline), 4,4′-sulfonylbis(aniline), polymethylene polyphenyl polyamines which comprise a mixture of methylene bridged polyphenyl polyamines containing from about 20 to about 90 percent by weight of methylenebis(aniline) and the remainder of the mixture being methylene bridged polyphenyl polyamines having a functionality greater than 2, and mixtures of the foregoing.

Preferred aromatic amines include: aniline, toluenediamine (including all isomers and mixtures of isomers), methylenebis(aniline) (including all isomers and mixtures of isomers), and mixtures thereof. Most preferred aromatic amines are 2,4-toluenediamine, 2,6-toluenediamine, 4,4′-methylenebis(aniline), 2,4′-methylenebis(aniline), and mixtures thereof.

Suitable urea compounds include N-aryl- substituted ureas and N,N′-diaryl- substituted ureas. Illustrative examples of ureas which can be employed include: N-phenylurea, N-(m-tolyl)urea, N-(p-tolyl)urea, N-phenyl-N′-methylurea, N-phenyl-N′-ethylurea, N-phenyl-N′-butylurea, N-phenyl-N′-hexylurea, N-phenyl-N′-benzylurea, N-phenyl-N′-phenethylurea, N-phenyl-N-cyclohexylurea, N,N′-diphenylurea, N,N′-di(m-tolyl)urea, N,N′-di(p-tolyl)urea. Preferred urea reactants are N,N′-diphenylurea, N,N′-di(m-tolyl)urea, and N,N′-di(p-tolyl)urea.

Additional suitable urea compounds include aromatic polyureas or aromatic polyurethane/ureas of the formula:

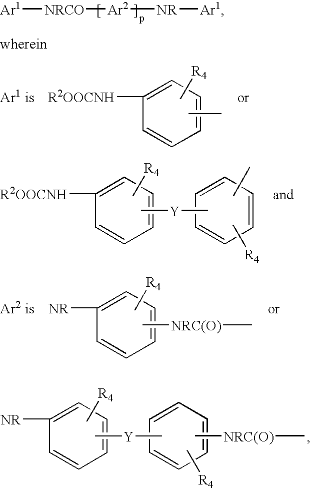

R independently each occurrence is hydrogen, alkyl, or aralkyl, preferably hydrogen;

R² independently each occurrence is hydrocarbyl of up to 20 carbons, preferably alkyl, such as methyl, ethyl, or butyl; and

p is an integer from 0 to 20, more preferably an integer from 0 to 4.

Preferred polyureas and polyuretheane/ureas have molecular weights less than 1,000,000, more preferably less than 10,000.

When applied to molecules which contain a plurality of urea linkages in a polymeric chain such as the process of treating polyureas and polyurethane/polyureas such as are obtained by reaction of a polyisocyanate and a polyamine or reaction of an isocyanate-terminated polyurethane prepolymer with a polyamine, the properties of the polymer are modified by shortening the chain length of said polymer and by introducing carbamate groups as terminal groups in the polymer chain. The latter groups can obviously be converted, as by acid hydrolysis of the ester and decarboxylation of the free carbamic acid, to the corresponding primary amino group thereby giving rise to an active center for further modification of the polymer.

The extent to which a polyurea or polyurethane/urea can be modified in the above manner is controlled by varying the amount of dialkyl carbonate employed in the reaction as well as by varying the time and temperature used in the treatment. If desired, complete degradation of the polyurea or polyurethane/polyurea can be achieved. That is substantially all the urea linkages in the polymer chain can be converted to carbamate functionality. Thus the process of the invention can be employed to recover scrap polyurea, or scrap polymer containing urea linkages, by converting the scrap to the corresponding carbamate compound from which the polymer was originally prepared.

The organic carbonates for use herein include dialkyl-, diaryl-, diaralkyl-, and cyclic alkylene esters of carbonic acid. Examples include, dimethyl carbonate, diethyl carbonate, dipropyl carbonate, dibutyl carbonate, diamyl carbonate, dihexyl carbonate, methyl ethyl carbonate, diphenyl carbonate, dibenzyl carbonate, ethylene carbonate, propylene carbonate, and mixtures thereof. Desired organic carbonates are those having up to 20 carbons. Preferred organic carbonates are the dialkyl carbonates, especially dimethyl carbonate, diethyl carbonate, dipropyl carbonate, and dibutyl carbonate.

The proportion in which the organic carbonate and the amine or urea containing reagents are employed is not critical to the process, excepting that to obtain complete conversion of amine or urea functionality, the organic carbonate should be present in at least a molar equivalency for each equivalent of amine or urea functionality present. Preferably the organic carbonate is employed in an excess to ensure complete conversion and to serve as a solvent for the reaction. Advantageously, the organic carbonate is employed in at least a 5 molar excess over the aromatic amine, and, preferably, in a range of from about 5 to about 30 moles of carbonate per mole of amine or urea.

Suitable heterogeneous catalysts comprise a Group 12-15 metal compound supported on a substrate, especially a porous support. Preferred metal compounds include derivatives of a Group 12, 14 or 15 compound, most preferably zinc, lead, or bismuth that are at least partially fixed to the exposed surface of a suitable support. Highly desirably, the metal compounds are relatively insoluble in the reaction mixture, even in the absence of the support. Suitable metal compounds include oxides, sulfides, carbonates, silicates, and nitrates of the foregoing metals, especially lead. A most preferred metal compound is PbO.

By the term “fixed” is meant the substrate provides a net coulombic attraction to the metal compound or physically absorbs the same thereby limiting the loss thereof during the reaction despite any solvating effect of the reaction mixture. The ability of the substrate to achieve the desired reduction in solubility or loss of catalyst may be determined by measuring the metal content in the reaction mixture, desirably under conditions of the reaction. Loadings of metal compound on the support may generally vary from 10 to 50 percent, preferably from 15 to 35 percent. Lower loadings generally give reduced activity whereas higher loadings result in loss of surface area and consequent loss of efficiency.

Preferred supports are organic or inorganic substances, including particulated materials or sintered solids, having surface areas ranging from 1 m²/g to 1000 m²/g, preferably from 50 m²/g to 300 m²/g. In measuring surface area herein the B.E.T. technique is one suitable method. Most preferably, the supports are in the form of pellets having a major dimension from 1 to 10 mm, preferably 1 to 5 mm. Preferred supports include carbon; organic or inorganic polymers, inorganic oxides, carbides, nitrides, or borides; and mixtures of the foregoing substrates. The supports may be in the form of particles, loose agglomerates or solid shapes such as spheres, pellets or sintered bars, rods or other masses. Preferred substrates include high surface area alumina, silica, aluminosilicate, aluminophosphate, and mixtures thereof. A most preferred substrate is alumina.

The catalyst may be prepared in one embodiment by contacting the metal compound or a precursor thereof, either neat or as a solution or mixture of the same with the substrate material. The resulting mixture may thereafter be treated in order to form the desired heterogeneous catalyst such as by converting the metal compound to a more stable or less fugitive form under the conditions of the reaction or to bond or otherwise fix the same to the substrate surface. Suitable treatments include heating the resulting material, optionally in the presence of an oxidizing agent, especially air or oxygen. A most preferred heterogeneous catalyst is lead oxide, PbO, generally formed by oxidation of Pb(NO₃)₂ or a soluble lead carboxylate such as lead di(2-ethylhexanoate) in situ on the surface of gamma alumina.

Preferably, the substrate is not completely devoid of surface hydroxyl or siloxy functional groups. In a particularly preferred embodiment, the support is high surface area alumina that has been calcined or heated at a temperature less than 800° C., preferably from 500 to 775° C., under conditions such that a portion of original surface hydroxyl functional groups are retained after such treatment. Suitable calcining conditions include heating in air or under nitrogen. Desirably the support is treated in the foregoing manner for a period from 30 minutes to 24 hours, more preferably from 1 to 5 hours prior to contacting with the Group 12-15 metal compound.

The heterogeneous catalyst may be employed in a loose packed bed comprising particles of substrate containing the metal compound on the surface thereof. The catalyst may also be compressed or sintered to form a larger mass while retaining significant porosity and surface area.

The present carbamate forming process may be carried out under reduced, elevated or atmospheric pressure and using relatively low reaction temperatures. Generally the reaction is conducted under sufficient pressure to maintain the reactants in a liquid phase and at temperatures of from 75 to 200° C., preferably from 100 to 190° C., and most preferably from 150 to 180° C.

The reactants can be mixed or combined in any order and heated to the desired reaction temperature in contact with the present heterogeneous catalysts until the desired degree of completion is attained. The extent completion of the reaction is easily determined using known standard analytical procedures to assay the disappearance of the reactants or maximum appearance of the desired carbamate product. Typical methods are infrared absorption analysis, gel permeation chromatography, gas phase chromatography, or high pressure liquid chromatography.

A particularly preferred means for carrying out the present invention comprises preheating a mixture of the aromatic amine or urea and the organic carbonate to a temperature of at least 50° C., preferably between 50 to 100° C., and passing the preheated mixture over a fixed bed comprising the heterogeneous supported catalyst. The process can be repeated any number of times or conducted in a continuous manner by passing the reaction mixture through a suitable fixed bed and continuously removing a product stream for separation of carbamate product. Inert diluents may be present in the reaction mixture, if desired. Suitable diluents include ethers such as tetrahydrofuran or diethyl ether, hydrocarbons, halogenated hydrocarbons and alcohols. A preferred diluent is tetrahydrofuran.

The carbamate products are isolated from the reaction mixture using standard separation procedures. Typically, the reaction solution is mixed with water and the carbamate is extracted from the aqueous solution using a water insoluble organic solvent, for example a halogenated solvent such as chloroform, carbon tetrachloride, or methylene dichloride. The organic solution is separated from the aqueous phase and the solvent removed using standard methods to provide the residual carbamate product. The carbamate, if desired, can be purified using standard methods such as recrystallization, column chromatography, or distillation.

As previously disclosed, the carbamate is desirably a derivative of an aromatic amine and is thermally decomposed to form the corresponding isocyanate as part of an integrated process for forming isocyanates from the corresponding aromatic amine and carbon monoxide. The process is particularly effective when the aromatic amine is a toluene diamine or mixture thereof, and the organic carbonate is dimethyl carbonate. Such an integrated process using 2,4-toluene diamine is conducted according to known process conditions and illustrated by the following scheme:

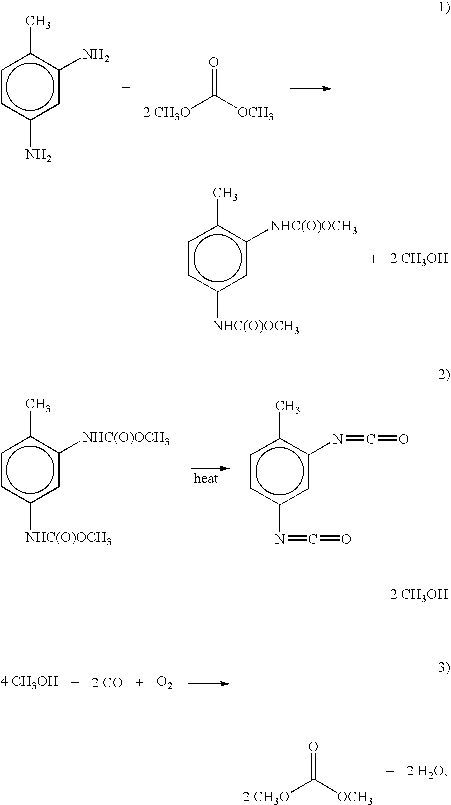

wherein the present invention is applied to step 1).

### SPECIFIC EMBODIMENTS

The following specific embodiments of the invention and combinations thereof are especially desirable and hereby delineated in order to provide detailed disclosure for the appended claims.

1. A process for the preparation of aromatic carbamates comprising contacting one or more organic carbonates with an aromatic amine or urea in the presence of a catalyst and recovering the resulting aromatic carbamate product, characterized in that the catalyst is a heterogeneous catalyst comprising a Group 12-15 metal compound supported on a substrate.

2. The process of embodiment 1 following the schematic formulas:

Ar(NRH)ᵣ+R′C(O)O [symbol] Ar(NRC(O)OR′)ᵣ+R′OH

Ar(NRC(O)NRR″)ᵣ+R′C(O)O [symbol] Ar(NRC(O)OR′)ᵣ+R″NRC(O)OR′,

wherein,

Ar is an aromatic or substituted aromatic group having a valency of r,

R independently each occurrence is hydrogen, alkyl, or aralkyl,

R′ independently each occurrence is alkyl or two R′ groups together are alkylene, and

R″ independently each occurrence is R or Ar.

3. The process of embodiment 1 or 2 wherein Ar independently each occurrence is selected from:

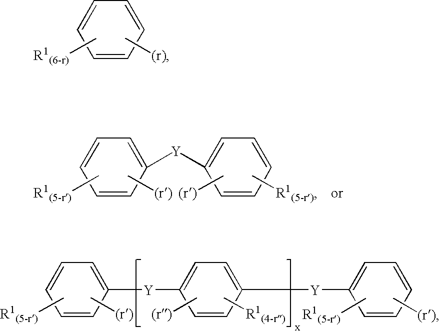

wherein R¹ independently each occurrence is hydrogen, halo, hydrocarbyl, inertly substituted hydrocarbyl, trihydrocarbylsilyl, trihydrocarbylsilylhydrocarbyl, or hydrocarbyloxy,

r is an integer greater than or equal to 1 which is equal to the valency of the aromatic group,

r′ individually each occurrence is an integer greater than or equal to 0 with the proviso that the sum of all r′ present (if no r″ is present) equals r,

r″ individually each occurrence is an integer greater than or equal to 0 with the proviso that where r″ is present, the sum x(r″)+all r′ equals r,

Y is selected from the group consisting of —O—, —CO—, —CH₂—, —SO₂—, —NR¹C(O)—, and a single bond, and

x is an integer greater than or equal to 0 indicating the number of repeating groups in the aromatic radical.

4. The process of any one of embodiments 1-3 wherein the aromatic amine is selected from the group consisting of aniline, p-methoxyaniline, p-chloroaniline, o-, m- or p-toluidine, 2,4-xylidine, 2,4-, and 2,6-toluenediamine, m- or p-phenylenediamine, 4,4′-diphenylenediamine, methylenebis(aniline) including 4,4′-methylenebis(aniline), 2,4′-methylenebis(aniline), 4,4′-oxybis(aniline), 4,4′-carbonylbis(aniline), 4,4′-sulfonylbis(aniline), polymethylene polyphenyl polyamines which comprise a mixture of methylene bridged polyphenyl polyamines containing from about 20 to about 90 percent by weight of methylenebis(aniline) and the remainder of the mixture being methylene bridged polyphenyl polyamines having a functionality greater than 2, and mixtures of the foregoing.

5. The process of any one of embodiments 1-4 wherein the aromatic amine is selected from the group consisting of aniline, toluenediamine (including all isomers and mixtures of isomers), methylenebis(aniline) (including all isomers and mixtures of isomers), and mixtures thereof.

6. The process of any one of embodiments 1-5 wherein the aromatic amine is selected from the group consisting of aniline, 2,4-toluenediamine, 2,6-toluenediamine, 4,4′-methylenebis(aniline), 2,4′-methylenebis(aniline), and mixtures thereof.

7. The process of any one of embodiments 1-6 wherein the urea compound is an N-aryl-substituted urea, a N,N′-diaryl- substituted urea, or an aromatic polyurea or aromatic polyurethane/urea of the formula:

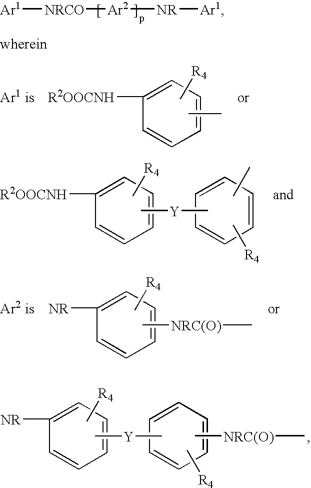

R independently each occurrence is hydrogen, alkyl, or aralkyl, preferably hydrogen;

R² independently each occurrence is hydrocarbyl of up to 20 carbons, preferably alkyl, such as methyl, ethyl, or butyl; and

p is an integer from 0 to 20, more preferably an integer from 0 to 4.

8. The process of any one of embodiments 1-7 wherein the urea compound is selected from the group consisting of N-phenylurea, N-(m-tolyl)urea, N-(p-tolyl)urea, N-phenyl-N′-methylurea, N-phenyl-N′-ethylurea, N-phenyl-N′-butylurea, N-phenyl-N′-hexylurea, N-phenyl-N′-benzylurea, N-phenyl-N′-phenethylurea, N-phenyl-N-cyclohexylurea, N,N′-diphenylurea, N,N′-di(m-tolyl)urea, N,N′-di(p-tolyl)urea, and mixtures thereof.

9. The process of any one of embodiments 1-8 wherein the urea compound is selected from the group consisting of N,N′-diphenylurea, N,N′-di(m-tolyl)urea, and N,N′-di(p-tolyl)urea.

10. The process of any one of embodiments 1-9 wherein the catalyst is PbO supported on alumina.

11. The process of any one of embodiments 1-10 wherein toluene diamine is converted to toluene di(methylcarbamate) by reaction with dimethylcarbonate.

12. A process for the formation of an isocyanate from an aromatic amine and carbon monoxide, said process comprising the steps of:

1) contacting a dialkyl carbonate with an aromatic amine under conditions to form an alkylcarbamate and an alcohol;

2) thermally decomposing the alkylcarbamate to form an aromatic isocyanate compound and an alcohol;

3) contacting the alcohol from step 1) and/or 2) with carbon monoxide under conditions to reform the dialkyl carbonate; and

4) recycling the dialkyl carbonate formed in step 3) for use in step 1) wherein the conditions of step 1) are those specified in any one of embodiments 1-9.

13. The process of embodiment 12 wherein the conditions of step 1) are those specified in any one of embodiments 1-11.

14. The process of any one of embodiments 11-13 comprising the following three unit operations:

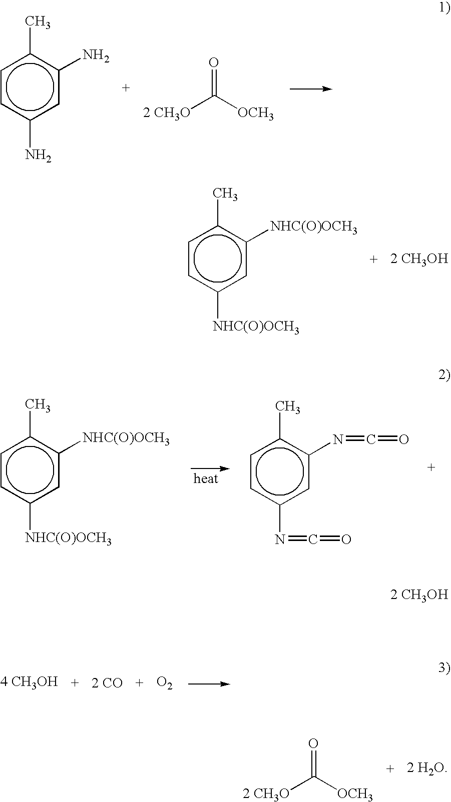

### EXAMPLES

It is understood that the present invention is operable in the absence of any component which has not been specifically disclosed. The following examples are provided in order to further illustrate the invention and are not to be construed as limiting. Unless stated to the contrary, all parts and percentages are expressed on a weight basis. The term “overnight”, if used, refers to a time of approximately 16-18 hours and “room temperature”, if used, refers to a temperature of 20-25° C.

The alumina is gamma alumina in the form of small relatively spherically shaped pellets, having a diameter of about ⅛″ (3 mm) (SAB-17™, available from Universal Oil Products Company (UOP)).

The fixed bed reactor consisted of a length of stainless steel tube of ⅜″ (9.5 mm) internal diameter having an internal volume of 35 mL which is loaded with the catalyst and placed in a forced air oven. Solvent (if any), aromatic amine supply, dimethyl carbonate supply, and nitrogen are connected via detachable feed lines to a feed supply tank. The oven temperature is controlled to ±1° C.

Aniline reaction products are analyzed by gas chromatography using nitrobenzene as the internal standard. Analyses are performed on a Hewlett Packard 6890 GC using a 30 meter DB-35 capillary column (0.53 mm id, 1.0 micron film thickness). Toluenediamine reaction products are analyzed by liquid chromatography using a C-18 column manufactured by Mac-Mod(Ace 5 C18 15 cm×4.6 mm with 5 μm particles) and optimized for the analysis of basic materials. Samples are prepared by dilution of about 90 microliters of reaction product with 3 mL of tetrahydrofuran, followed by filtration of the sample before injection. Triethylamine is added to the aqueous and organic phases to obtain the best peak shape. The amine reacts with any underivatized silanol groups to prevent tailing of the analyte. The column is run at room temperature with a 1 ml/min flow rate and the following gradient: 90 percent water, 10 percent acetonitrile to 10 percent water, 90 percent acetonitrile in 20 minutes. The reaction products are detected with a UV detector operating at 235 nm.

### Example 1

### Aniline Conversion Using 10 Percent PbO on Alumina

Lead (II) nitrate (2.0 g) is dissolved in deionized water (25 mL) and added to 12.5 g of alumina. The catalyst is air dried at room temperature for 24 hours, then calcined at 500° C. in air for 4 hours, under which conditions the lead nitrate is converted to PbO.

The fixed bed reactor is loaded with 34 mL, 10.2 g of the above-prepared catalyst. A feed mixture of aniline (5 parts), tetrahydrofuran (THF) (50 parts) and dimethyl carbonate (DMC) (45 parts) is prepared. The reactor is heated to 180° C. with a pressure set point of 200 psig (1.5 MPa) and a feed rate of 0.5 mL/min. After 25 hours of operation the aniline conversion is 45 percent with 94 percent selectivity to methyl N-phenyl carbamate and phenyl isocyanate and 6 percent selectivity to N-methyl aniline.

### Example 2

### TDA Conversion Using 30 Percent PbO on Alumina

A sample of alumina (10.0 g) is impregnated with lead (II) nitrate (6.9 g) dissolved in deionized water (20.0 g). The impregnated beads are dried in air overnight, heated at 150° C. for 3 hours and calcined at 500° C. for 16 hours in air. The catalyst (14.2 g, 35 mL) is loaded into the fixed bed reactor and heated to a temperature of 160° C. A mixture of 2.6 parts 2,4-toluene diamine (TDA), 40 parts THF, and 57.4 parts DMC is passed through the reactor at 0.5 mL/min., achieving an amine conversion of approximately 80 percent with 90 selectivity to the mono and dicarbamate products.

### Example 3

### TDA Conversion Using 30 Percent PbO on Alumina

Alumina (10.0 g) is impregnated with lead (II) nitrate (6.9 g) dissolved in deionized water (20.0 g). The impregnated beads are dried in air overnight, heated at 150° C. for 3 hours and calcined at 750° C. for 4 hours in air. The catalyst is loaded into the fixed bed reactor and evaluated over a period of almost 570 hours using a mixture of 3 parts TDA, 30 parts THF and 67 parts DMC. The temperatures, pressures, feed rates and feed times used are:

160° C., 120 psig (930 kPa), 0.4 mL/min, 0-65 hrs

160° C., 120 psig (930 kPa), 0.3 mL/min, 65-135 hrs

160° C., 120 psig (930 kPa), 0.4 mL/min, 135-180 hrs

165° C., 120 psig (930 kPa), 0.4 mL/min, 184-372 hrs

165° C., 120 psig (930 kPa), 0.3 mL/min, 375-545 hrs

170° C., 150 psig (1.1 MPa), 0.3 mL/min, 452-500 hrs

160° C., 150 psig (1.1 MPa), 0.3 mL/min, 517-568 hrs

Initial activity is more than 90 percent amine conversion with mono and dicarbamate selectivity of almost 95 percent. With time on stream the amine conversion and carbamate selectivity slowly decline. By 180 hours the amine conversion drops to about 50 percent and the total carbamate selectivity to just less than 90 percent.

A product sample taken during the first 40 hours on stream is analyzed by XRF spectroscopy and found to contain 46 ppm lead. A product sample taken after 500 hours operation and similarly analyzed shows no detectable lead content.

### Example 4

### TDA Conversion Using PbAl₂O₄ on Alumina

Lead nitrate (6.9 g) is dissolved in water (20.5 g) and then added to 10.1 g of alumina. The impregnated alumina is vacuum dried at 55° C. then calcined at 825° C. in air for 15 hours. Analysis by X-ray powder diffraction is consistent with the formation of lead aluminate (PbAl₂O₄). A portion of the calcined catalyst (35 mL, 13.1 g) is loaded into the fixed bed reactor. A solution of 4.3 parts TDA, and 95.7 parts DMC is passed through the catalyst bed at 160° C., at 120 psig (930 kPa) and a flow rate of 0.32 mL/min. The initial amine conversion is approximately 80 percent, with total carbamate selectivity between 60 and 80 percent. Both conversion and selectivity decline with continued operation.

### Example 5

### TDA Conversion Using 30 Percent PbO on Carbon

Carbon particles (18 g, 12×20 mesh) are impregnated with lead (II) nitrate (11.5 g) dissolved in deionized water (27 g). After air drying for 24 hours, the carbon sample is heated in a tube furnace, under nitrogen flow to 500° C. to decompose the lead nitrate providing a calculated loading of about 30 percent PbO. The catalyst (17.6 g) is loaded into the fixed bed reactor and evaluated at 170° C., 150 psig (1.1 MPa) and 0.32 mL/min flow rate using a reaction mixture of 3 parts TDA, 30.2 parts THF and 66.8 parts DMC. The amine conversion starts at nearly 98 percent and slowly declines to 88-92 percent over 200 hours of operation. At the same time total carbamate selectivity declines from about 50 percent to about 25 percent.

### Example 6

### TDA Conversion Using 30 Percent Bi₂O₃ on Alumina

A solution of 10 g of bismuth trioxide (Hex CEM™ 32 percent Bi, available from OM Group) in toluene (9.0 g) is added dropwise to 8.0 g of alumina. The impregnated beads are dried in air to constant weight and then calcined at 500° C. for 6 hours in air. The product (8.0 g, 21 mL) is loaded into the fixed bed reactor and evaluated with a mixture of 4.3 parts TDA and 95.7 parts DMC. Reaction conditions are 165° C., 120 psig (930 kPa), 0.25 mL/minute flow for the first 30 hours and 160° C., 120 psig (930 kPa), 0.25 mL/minute flow for hours 30-200. Amine conversion is about 80 percent with carbamate selectivity of 90 percent initially, declining to about 50 percent conversion and 70 percent selectivity after 200 hours of operation.

### Example 7

### TDA Conversion Using PbO on Zinc Oxide

A sample of zinc oxide pellets (Zn 0101™ available from Engelhard Corporation) is exhaustively washed with water and then air-dried. Lead (II) di(2-ethylhexanoate) (7.7 g of 55 percent Pb(O₂C₈H₁₅)₂ in mineral spirits) is added to the washed zinc oxide (39.1 g). The pellets are mixed to thoroughly wet the pellets and then are collected by filtration and washed with toluene. The pellets are transferred to a petri dish where they are allowed to air dry and then calcined at 500° C. for four hours in air. The PbO/ZnO catalyst (39.7 g, 26 mL) is loaded into the fixed bed reactor and evaluated using a mixture of 4.3 parts TDA and 95.7 parts DMC under the following conditions:

160 C, 150 psig (1.1 Mpa), 0.32 mL/min, 0-41 hrs

170 C, 150 psig (1.1 Mpa), 0.32 mL/min, 41-67 hrs

180 C, 150 psig (1.1 Mpa), 0.20 mL/min, 67-71 hrs

170 C, 150 psig (1.1 Mpa), 0.20 mL/min, 71-90 hrs

180 C, 175 psig (1.3 Mpa), 0.20 mL/min, 90-135 hrs

180 C, 175 psig (1.3 Mpa), 0.20 mL/min, 135-326 hrs

Initial activity is 45 percent amine conversion and 70 percent total carbamate selectivity increasing over 60 hours to 98 percent conversion and 70 percent selectivity. After 326 hours of operation amine conversion remains about 80 percent with 60 percent selectivity to mono and dicarbamate products.

### Comparative A TDA Conversion Using Zinc Oxide

A sample of 49 g, 30 mL of zinc oxide is loaded in to the fixed bed reactor and heated to 170° C. A mixture of 4.3 parts TDA and 95.7 parts DMC is passed through the reactor at a pressure of 150 psig (1.3 MPa) 0.32 mL/min. for 52 hours. Amine conversion briefly peaks at 90 percent at 25 hours of operation and falls to 25 percent after 50 hours operation. Selectivity to mono and dicarbamates reaches no higher than 50 percent.

### Comparative B TDA Conversion Using Alumina

The fixed bed reactor is loaded with 10.3 g, 34 mL of alumina. A mixture of 4.3 parts TDA and 95.7 parts DMC is passed through the reactor over 70 hours under the following conditions:

160 C, 150 psig (1.1 MPa), 0.32 mL/min, 0-47 hrs

160 C, 85 psig (690 kPa), 0.32 mL/min, 47-66 hrs

160 C, 197 psig (1.5 MPa), 0.32 mL/min, 66-70 hrs

Initially the amine conversion is about 75 percent with about 45 percent total carbamate selectivity. After 70 hours of operation, amine conversion drops to about 65 percent with carbamate selectivity of approximately 65 percent.

1. A process for the preparation of aromatic carbamates comprising contacting one or more organic carbonates with an aromatic amine or urea in the presence of a catalyst and recovering the resulting aromatic carbamate product, characterized in that the catalyst is a heterogeneous catalyst comprising a Group 12-15 metal compound supported on a substrate.

2. The process of claim 1 following the schematic formulas:
Ar(NRH)ᵣ+R′OC(O)O [symbol] Ar(NRC(O)OR′)ᵣ+R′OH
Ar(NRC(O)NRR″)ᵣ+R′OC(O)O [symbol] Ar(NRC(O)OR′)ᵣ+R″NRC(O)OR′,
wherein,
Ar is an aromatic or substituted aromatic group having a valency of r,
R independently each occurrence is hydrogen, alkyl, or aralkyl,
R′ independently each occurrence is alkyl or two R′ groups together are alkylene, and
R″ independently each occurrence is R or Ar.

3. The process of claim 2 wherein Ar independently each occurrence is selected from: 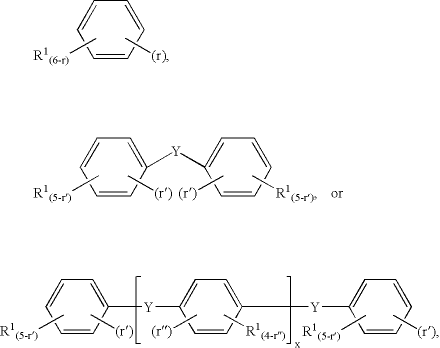
wherein R¹ independently each occurrence is hydrogen, halo, hydrocarbyl, inertly substituted hydrocarbyl, trihydrocarbylsilyl, trihydrocarbylsilylhydrocarbyl, or hydrocarbyloxy,
r is an integer greater than or equal to 1 which is equal to the valency of the aromatic group,
r′ individually each occurrence is an integer greater than or equal to 0 with the proviso that the sum of all r′ present (if no r″ is present) equals r,
r″ individually each occurrence is an integer greater than or equal to 0 with the proviso that where r″ is present, the sum x(r″)+all r′ equals r,
Y is selected from the group consisting of —O—, —CO—, —CH₂—, —SO₂—, —NR¹C(O)—, and a single bond, and
x is an integer greater than or equal to 0 indicating the number of repeating groups in the aromatic radical.

4. The process of claim 1 wherein the aromatic amine is selected from the group consisting of aniline, p-methoxyaniline, p-chloroaniline, o-, m- or p-toluidine, 2,4-xylidine, 2,4-, and 2,6-toluenediamine, m- or p-phenylenediamine, 4,4′-diphenylenediamine, methylenebis(aniline), 2,4′-methylenebis(aniline), 4,4′-oxybis(aniline), 4,4′-carbonylbis(aniline), 4,4′-sulfonylbis(aniline), polymethylene polyphenyl polyamines, and mixtures of the foregoing.

5. The process of claim 1 wherein the aromatic amine is selected from the group consisting of aniline, toluenediamine, methylenebis(aniline), and mixtures thereof.

6. The process of claim 1 wherein the aromatic amine is selected from the group consisting of aniline, 2,4-toluenediamine, 2,6-toluenediamine, 4,4′-methylenebis(aniline), 2,4′-methylenebis(aniline), and mixtures thereof.

7. The process of claim 1 wherein the urea compound is an N-aryl- substituted urea, a N,N′-diaryl- substituted urea, or an aromatic polyurea or aromatic polyurethane/urea of the formula: 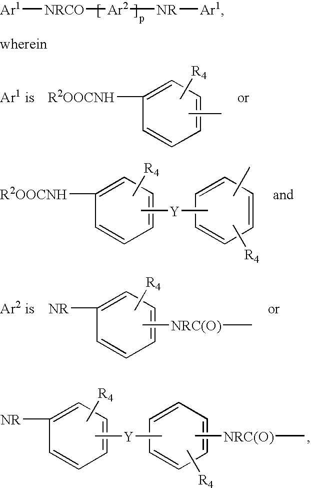
R independently each occurrence is hydrogen, alkyl, or aralkyl;
R² independently each occurrence is hydrocarbyl of up to 20 carbons; and
p is an integer from 0 to 20.

8. The process of claim 1 wherein the urea compound is selected from the group consisting of N-phenylurea, N-(m-tolyl)urea, N-(p-tolyl)urea, N-phenyl-N′-methylurea, N-phenyl-N′-ethylurea, N-phenyl-N′-butylurea, N-phenyl-N′-hexylurea, N-phenyl-N′-benzylurea, N-phenyl-N′-phenethylurea, N-phenyl-N-cyclohexylurea, N,N′-diphenylurea, N,N′-di(m-tolyl)urea, N,N′-di(p-tolyl)urea, and mixtures thereof.

9. The process of claim 1 wherein the urea compound is selected from the group consisting of N,N′-diphenylurea, N,N′-di(m-tolyl)urea, and N,N′-di(p-tolyl)urea.

10. The process of any one of claims 1-9 wherein the catalyst is PbO supported on alumina.

11. The process of claim 1 wherein toluene diamine is converted to toluene di(methylcarbamate) by reaction with dimethylcarbonate.

12. A process for the formation of an isocyanate from an aromatic amine and carbon monoxide, said process comprising the steps of:
1) contacting a dialkyl carbonate with an aromatic amine under conditions to form an alkylcarbamate and an alcohol;
2) thermally decomposing the alkylcarbamate to form an aromatic isocynate compound and an alcohol;
3) contacting the alcohol from step 1) and/or 2) with carbon monoxide under conditions to reform the dialkyl carbonate; and
4) recycling the dialkyl carbonate formed in step 3) for use in step 1) wherein the conditions of step 1) are those specified in any one of claims 1-9;

13. The process of claim 12 wherein the conditions of step 1) are those specified in claim 11.

14. The process of claim 12 comprising the following three unit operations: [chemical structure]
